(19) United States
(12) Patent Application Publication
(10) Pub. No.: US 2017/0081701 A1
(43) Pub. Date: Mar. 23, 2017

(54) sPLA2 MONITORING STRIP

(71) Applicant: Drexel University, Philadelphia, PA (US)

(72) Inventors: Timothy J. Cunningham, Wyncote, PA (US); Katherine Marie Kollins Callaghan, Southampton, PA (US); Lihua Yao, Wynnewood, PA (US)

(73) Assignee: Drexel University, Philadelphia, PA (US)

(21) Appl. No.: 15/366,317

(22) Filed: Dec. 1, 2016

Related U.S. Application Data
(63) Continuation of application No. 14/124,430, filed on Jun. 20, 2014, now abandoned.
(60) Provisional application No. 61/494,121, filed on Jun. 7, 2011.

(51) Int. Cl. C12Q 1/44

(52) U.S. Cl. CPC C12Q 1/44; C12Y 301/01004; G01N 2333/92; G01N 2405/04

(57) ABSTRACT

A device and method for determining the presence or absence, or the level of, sPLA2 activity in a fluid sample. The device includes an absorbent matrix that defines a flow path for a fluid sample, a first region of the absorbent matrix for applying a fluid sample, where one of the components selected from a bioactive sPLA2 substrate and a label is dried onto or within the first region of the absorbent matrix, a second region of the absorbent matrix downstream of, and in fluid communication with, the first region for detecting an aggregated reaction product, where the other component not present in the first region is dried onto or within the second region of the absorbent matrix.

### CROSS-REFERENCE TO RELATED APPLICATIONS

This application claims priority to U.S. Provisional Application No. 61/494,121 filed Jun. 7, 2011, which is hereby incorporated herein by reference in its entirety.

### BACKGROUND OF THE INVENTION

Phospholipase A2 (PLA2) catalyzes the hydrolysis of phospholipids at the sn-2 position, yielding a free fatty acid and a lysophospholipid. The release of arachidonic acid from membrane phospholipids by PLA2 is believed to be a key step in the control of eicosanoid production within the cell.

More recently, type II secretory phospholipase A2 (sPLA2) has been recognized as an independent predictor of cardiovascular events. For example, it is postulated that the tissue expression of sPLA2 is one of the links between inflammatory processes and lipid accumulation in atherosclerosis. This enzyme is also present in the media of normal and diseased arteries, and hydrolyzes phospholipids at the sn-2 position generating lysophospholipds and fatty acids. The local production of oxidized low density lipoprotein results in enhanced uptake by macrophages and subsequent transformation into foam cells. sPLA2 exhibits similar features like CRP as a marker of plaque inflammation. This includes its positive association with cardiovascular risk factors, the link between enhanced plasma levels (245 ng/dl) and the occurrence of coronary events both in patients with stable and unstable angina (Kugiyama et al., 2000, Am J Cardiol 86:718-722).

Thus, sPLA2 activity both reflects and drives several inflammatory disorders, and elevations of this activity may signal flare ups in diseases like multiple sclerosis, rheumatoid arthritis and others. There is thus a long-felt need in the art for a convenient, user friendly device to monitor levels of sPLA2 activity, thereby providing a novel mechanism for monitoring pathology and response to treatment for a variety of inflammatory diseases. The present invention satisfies this need.

### SUMMARY OF THE INVENTION

The invention provides a device for detecting the presence or absence of sPLA2 in a fluid sample. In one embodiment, the device comprises an absorbent matrix that defines a flow path for a fluid sample; a first region of the absorbent matrix for applying a fluid sample, wherein one of the components selected from a bioactive sPLA2 substrate and a label is dried onto or within the first region of the absorbent matrix; and a second region of the absorbent matrix downstream of, and in fluid communication with, the first region for detecting an aggregated reaction product, wherein the other component not present in the first region is dried onto or within the second region of the absorbent matrix; wherein in the absence of sPLA2 in the fluid sample, applying the fluid sample does not result in a recognizable aggregated reaction product in the second region; and wherein in the presence of sPLA2 in the fluid sample, applying the fluid sample results in a detectable aggregated reaction product in the second region.

In one embodiment, the label comprises a gold sol.

In one embodiment, the gold sol comprises streptavidin coated gold particles.

In one embodiment, the bioactive sPLA2 substrate is Diheptanoyl Thio-PC.

In one embodiment, the device further comprises a linker molecule dried onto or within the absorbent matrix in the same region as the bioactive sPLA2 substrate.

In one embodiment, the linker molecule is biotin-maleimide.

In one embodiment, the blocker molecule is dried onto or within the absorbent matrix in the same region as the linker molecule.

In one embodiment, the fluid sample is a biological sample.

In one embodiment, a biological sample is urine.

The invention also provides a device for determining the level of sPLA2 activity in a fluid sample. In one embodiment, the device comprises an absorbent matrix that defines a flow path for a fluid sample; a first region of the absorbent matrix for applying a fluid sample, wherein one of the components selected from a bioactive sPLA2 substrate and a label is dried onto or within the first region of the absorbent matrix; a second region of the absorbent matrix downstream of, and in fluid communication with, the first region for detecting an aggregated reaction product, wherein the other component not present in the first region is dried onto or within the second region of the absorbent matrix; wherein after applying a fluid sample containing sPLA2 to the first region, one of the color or intensity of the aggregated reaction product can be compared to a predetermined set of colors or intensities, wherein each of the predetermined colors or intensities is indicative of a level of sPLA2 activity in the fluid sample.

In one embodiment, the label comprises a gold sol.

In one embodiment, the gold sol comprises streptavidin coated gold particles.

In one embodiment, the bioactive sPLA2 substrate is Diheptanoyl Thio-PC.

In one embodiment, the device further comprises a linker molecule dried onto or within the absorbent matrix in the same region as the bioactive sPLA2 substrate.

In one embodiment, the linker molecule is biotin-maleimide.

In one embodiment, a blocker molecule is dried onto or within the absorbent matrix in the same region as the linker molecule.

In one embodiment, the fluid sample is a biological sample.

In one embodiment, the biological sample is urine.

The invention also provides a device for determining the level of sPLA2 activity in a fluid sample. In one embodiment, the device comprises an absorbent matrix that defines a flow path for a fluid sample; a first region of the absorbent matrix for applying a fluid sample, wherein one of the components selected from a bioactive sPLA2 substrate and a label is dried onto or within the first region of the absorbent matrix; a second region of the absorbent matrix downstream of, and in fluid communication with, the first region, wherein the other component not present in the first region is dried onto or within the second region of the absorbent matrix; and a third region of the absorbent matrix downstream of the first region, and in fluid communication with the first and second regions, for detecting an aggregated reaction product; wherein after applying a fluid sample containing sPLA2 to the first region, the liquid sample mobilizes the components of the first and second regions and forms an detectable aggregation product in the third region, and wherein one of the color or intensity of the aggregated reaction product can be compared to a predetermined set of colors or intensities, wherein each of the predetermined colors or intensities is indicative of a level of sPLA2 activity in the fluid sample.

The invention also provides a method of determining the presence or absence of sPLA2 in a fluid sample comprising adding a fluid sample to the device of the invention; allowing the fluid sample to flow along the flow path to form a detectable aggregated reaction product in the second region when sPLA2 is in the fluid sample; and observing the second region of the device to determine the presence or absence of a detectable aggregated reaction product.

The invention also provides a method of determining a level of sPLA2 activity in a fluid sample comprising adding a fluid sample to the device of the invention; allowing the fluid sample to flow along the flow path to form a visibly detectable aggregated reaction product in the detecting region when sPLA2 is in the fluid sample; observing the detecting region of the device to determine at least one of the color or intensity of the visibly detectable aggregated reaction product; and comparing at least one of the color or intensity of the visibly detectable aggregated reaction product to a predetermined set of colors or intensities, wherein each of the predetermined colors or intensities is indicative of a level of sPLA2 activity in the fluid sample.

The invention also provides a method of determining a pathology, a disease or response to treatment of a disease, comprising adding a fluid sample collected from a patient to the device of the invention; allowing the fluid sample to flow along the flow path to form a visibly detectable aggregated reaction product in the detecting region when sPLA2 is in the fluid sample; observing the detecting region of the device to determine at least one of the color or intensity of the visibly detectable aggregated reaction product; and comparing at least one of the color or intensity of the visibly detectable aggregated reaction product to a predetermined set of colors or intensities, wherein each of the predetermined colors or intensities is indicative of a pathology, a disease, or of a category of pathology or disease in the patient.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is an image of an exemplary device for monitoring levels of sPLA2 activity. The device includes a bottom or base layer of nitrocellulose NC90 and a top layer of Whatman Fusion 5 paper, with the ends sealed by rubber cement. The device includes three designated regions. The first region includes a dried mixture of bioactive sPLA2 substrate, an optional linker molecule with a biotin moiety (biotin-maleimide), and an optional blocking compound (L-cysteine-agarose). The second region comprises a dried colloidal gold particle which may optionally be covalently linked to streptavidin. The third region is a detection zone located between the first and second regions.

FIG. 2, comprising FIGS. 2A-2C, is a photo image of three devices (2A-C) constructed according to the embodiment of FIG. 1. For each of devices 2A-C, the first region was infused with 10 μl of a mixture of 25% 48 mM bis-thioPC substrate in 10 mM Tris-150 mM NaCl buffer, pH=7.4 that contained 4 mM CaCl₂ and 10 μg maleimide-biotin. Care was taken to restrict fluid to the first region area only. The pads were dried 15 min under vacuum. Cysteine solution (7 μl, 1.5 mM) was added to the first region and air dried. Nanogold (40 nM, optical density 15) was used without dilution and 2.5 μl was delivered in 0.5 μl spots in a strip across the second region of each device 2A-C. The paper for each was air-dried and the procedure was repeated twice. A first control strip (2B) was prepared in which the substrate's vehicle was used instead of substrate (no substrate), and a second control strip (2C) was prepared in which the enzyme's vehicle was added during the test instead of enzyme (no enzyme). The samples for devices 2A and 2B were 5×10⁻⁸M sPLA2 added as two large drops over the first region. The sample for 2C only contained the enzyme vehicle. A dark blue reaction appears in the third region only when enzyme and substrate are both present as depicted in device 2A.

FIG. 3 is an image of a second exemplary device for monitoring levels of sPLA2 activity. The device includes a bottom or substrate layer of nitrocellulose NC90 and a top layer of Whatman Fusion 5 paper covering a portion of the base layer, with the end sealed by rubber cement. The device includes two designated regions. The first region (the top layer of Whatman Fusion 5 paper) was spotted with colloidal gold (40 nM, optical density (OD) 15 used without dilution, or OD 50 used at 25% in water) by delivering about 10-15 μl in 1 μl spots over the entire region then air-dried. The second region (exposed substrate layer) was spotted with 10 μl of a mixture of 25% 48 mM bis-thio PC substrate in 10 mM Tris-150 mM NaCl buffer, pH=7.4 that contained 4 mM CaCl₂ and vacuum dried.

FIG. 4, comprising FIGS. 4A and 4B, is a photo image of two devices (4A and 4B) constructed according to the embodiment of FIG. 3. The size of each device is ˜1.5×3 cm. One drop of human urine was added to the first region of each device 4A and 4B from a Pasteur pipette. Dark red lines appeared in the second region and a thin line appeared at the border with the first region of device 4B. The control strip (4A) without substrate is also shown. Controls without enzyme were also non-reactive.

FIG. 5 depicts the sensor mechanism of the devices of FIGS. 1-4. The portion of the visible spectrum of the light reflected from the gold particles depends on surface plasmon resonance which in turn depends on size, attachments, and extent the particles are aggregated (Lee et al., 2006, J Phys Chem B 110(39):19220-5). Without wishing to be bound by any particular theory, it is believed that the amount of fatty acid cleaved from the sPLA2 substrate (enzyme reaction product) dictates the extent of aggregation. As activity increases, aggregation increases, and the color changes from pink (low aggregation) to red (medium aggregation) to blue (high aggregation), respectively.

FIG. 6 depicts a proposed molecular mechanism of the present invention. The gold particles are mobilized by addition of the sample and flow from the first region to the second region, which is where they encounter sPLA2 substrate. Cleavage of the A2 fatty acid bond releases thiols that attach to the particles. Particle aggregation and color change occurs with the natural affinities between hydrophobic fatty acids, not unlike the mechanism that underlies micelle formation by phospholipids in aqueous solutions.

FIG. 7 depicts an exemplary test strip that has no amplification or blocking reagents. This test strip also has a separate sample pad.

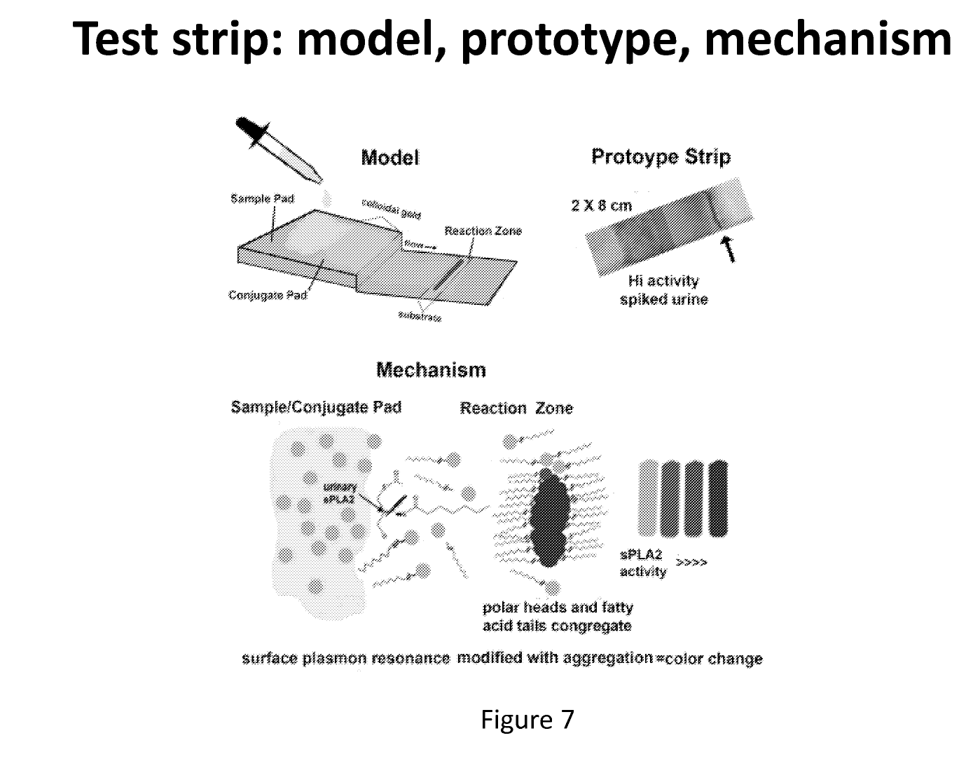

FIG. 8 depicts an exemplary dipstick.

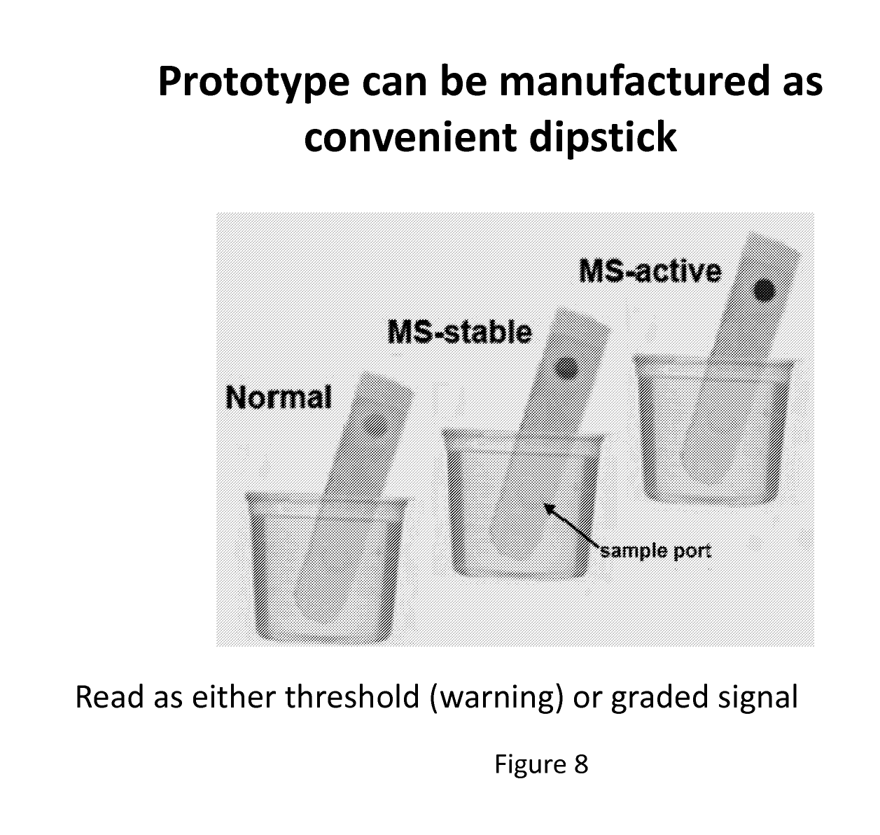

FIG. 9 depicts an alternative device for monitoring levels of sPLA2 activity.

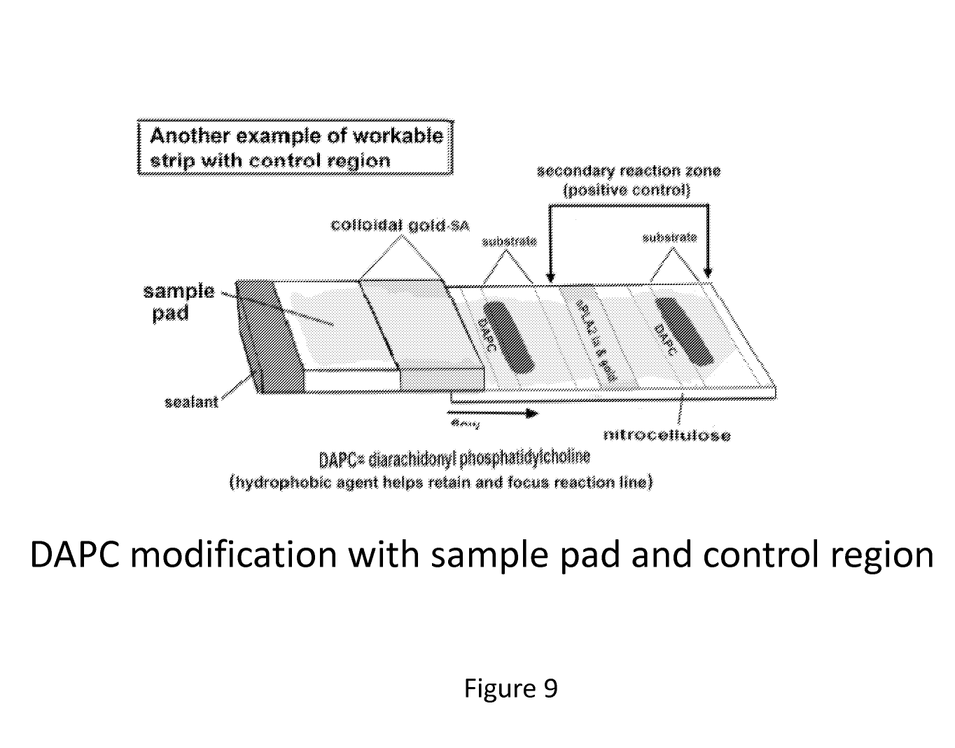

FIG. 10 depicts regions on a representative device where sensitivity adjustments, targeting specific patient population, can be made. Region 1 depicts the sample pad region which acts as scavengers for interfering solutes (overall sensitivity). For example, protein A/G and maleimide beads for Ig and for sulphydryls respectively. Region 2 depicts the indicator pad where the size and density of gold particles (aggregate size, scaling) as well as stabilizing and/or blocking agents (e.g., gold affinity for SH-fatty acid) can be adjusted. For example, either about 30 or 40 nM particles can be used and “naked” particles can be blocked with about 0.03% tween 20 or strepavidin coated particles with no detergent. Region 3 depicts the reaction zone. For example, Region 3 can comprise mixed liposome substrates (enzyme-substrate affinity) on Nc 90 paper from Millipore. In one embodiment, Region 3 comprises mixed liposomes 1:1 Diheptanoyl thio-PC (substrate) with dioleoyl phosphatidyl serine in buffer with 0.1% triton X 100. Region 4 depicts a wicking strip having a paper speed (enzyme dwell time in reaction zone). For example, Nc 240 paper from Millipore. In some instances, it is not necessary to have the wicking paper.

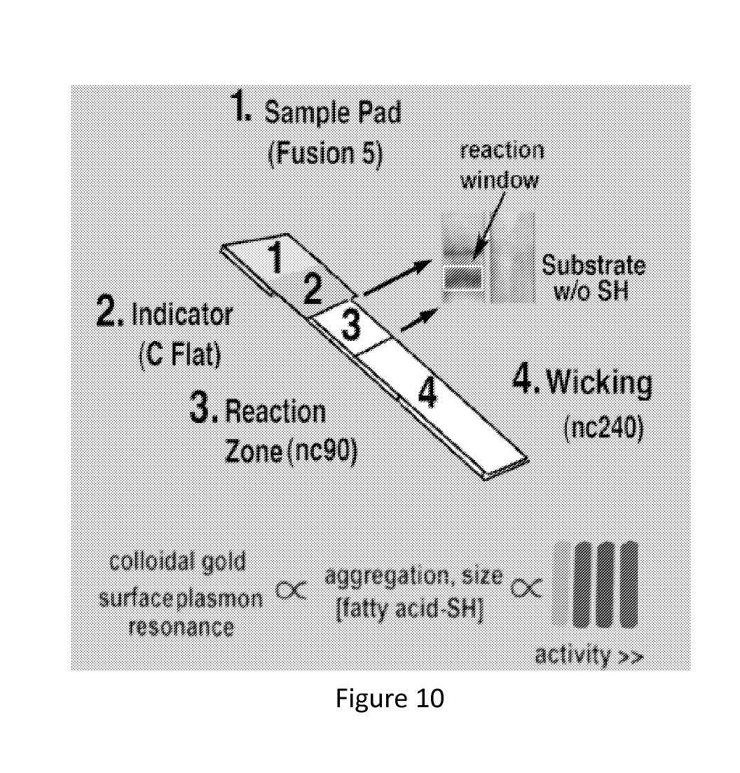

### DETAILED DESCRIPTION

The present invention relates to devices and methods for conveniently monitoring the presence or absence of sPLA2 activity, as well as determining variable levels of sPLA2 activity, when present. The device may take the form of a user-friendly reactant strip having a sample application region and a readable detection region to indicate sPLA2 activity. For example, the present invention allows a user to monitor levels of secreted phospholipase A2 (sPLA2) activity in in a liquid sample, such as urine. These devices and methodologies provide a new and convenient mechanism for monitoring pathology and response to treatment for a variety of inflammatory diseases including multiple sclerosis, rheumatoid arthritis, atherosclerosis, and Alzheimer's disease.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate. Non-limiting examples of diseases that can be monitored by the present invention include inflammatory diseases, such as multiple sclerosis, rheumatoid arthritis, atherosclerosis and Alzheimer's disease.

As used herein, an “instructional material” includes a publication, a recording, a diagram, or any other medium of expression which can be used to communicate the usefulness of a component of the invention in a kit for monitoring sPLA2 activity as recited herein. Optionally or alternately, the instructional material can describe one or more methods of monitoring an inflammatory disease based on sPLA2 activity as recited herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “sample” as used herein, refers to a biological sample from a subject, including but is not limited to tissue, blood, saliva, feces, and urine. A sample can also be any other source of material obtained from a subject which contains a compound or cells of interest. A “liquid sample” means the sample is flowable through an absorbent material. The liquid sample may be a liquid biological sample, or it may be a biological sample suspended in any suitable fluid.

“Aggregation” means a massing together or clustering of independent but similar units, such as particles, parts, or bodies.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to devices and methods for conveniently monitoring the presence or absence of sPLA2 activity, as well as determining variable levels of sPLA2 activity, when present. The device may include a base or support layer and an absorbent matrix composed of at least one absorbent layer through which a liquid sample can flow along a flow path by force or by capillary action. The base layer may also be absorbent and be in fluid communication with the absorbent matrix, such that the flow path of liquid sample passes through both the absorbent matrix and the base layer. The flow path includes at least two regions, where the first region is a sample application region, and the second region is a detection region. One of the regions contains a bioactive sPLA2 substrate, while the other region contains a label, such as a colloidal gold particle. In operation, a sample containing sPLA2 enzyme is added to the sample loading region and flows along a path through the absorbent matrix. The enzyme cleaves the A2 fatty acid bond, releasing fatty acid thiols that attach to the label. These labeled fatty acid thiols aggregate within the detection region, where they undergo a color change based on the size and concentration of the labeled fatty acid aggregation, and thereby corresponding to level of enzyme activity. When colloidal gold is used, a lower activity is indicated by a pink color, while a higher activity is indicated by a darker red or blue color. If the label is located in the sample loading region, the label is mobilized when sample is added, and travels with the liquid sample to the detection region, where the bioactive sPLA2 substrate is located. If the bioactive sPLA2 substrate is located in the sample loading region, then the fatty acid thiols are cleaved and mobilized within the liquid sample flow path and into the detection region, where the label is located. In some instances, the device may include three regions, where the first region is the sample loading region and contains one of the bioactive sPLA2 substrate or the label, the second region contains the other of the bioactive sPLA2 substrate or the label, and the third region is the detection region, which may be positioned between the first and second regions, or it may be downstream of the first and second regions. The present invention also provides a method of monitoring pathology and response to treatment of an inflammatory disease, including (without limitation) multiple sclerosis, rheumatoid arthritis, atherosclerosis and Alzheimer's disease. The method includes the steps of monitoring sPLA2 activity in a liquid sample, where increased sPLA2 activity is indicative of the disease or a flare up of the disease.

### Device Construction

Generally, the device may take the form of a strip or a circular “bullseye”, and includes an absorbent matrix composed of at least one absorbent layer, whereby a flow path (such as a lateral flow path) of a liquid sample can be defined through the absorbent matrix either by force or by capillary action. The absorbent matrix may be a single layer composed of a single material, or it may be a single layer composed of multiple materials, or separate materials, whereby the separate materials are in fluid communication with each other along the flow path. Further, the absorbent layer may include absorbent layers of different lengths, whereby the material of one layer may be a separate material of another layer. Nonetheless, each material forming part of the flow path will be in fluid communication with each other. The materials of the absorbent matrix can be any material that is porous or absorbs fluid and allows for transport of a fluid sample therethrough. Non-limiting examples of such materials are Whatman Fusion 5 paper, nitriocellulose, nitrocellulose, filter papers, glass fibers, polyester, and other suitable materials as would be understood by those skilled in the art.

The device may optionally include a base or support substrate layer, upon which the absorbent matrix is positioned on top of. In embodiments where the absorbent matrix is composed of multiple material components, layers or sections, the presence of a base layer may provide a solid substrate on which to build the absorbent matrix, thereby creating a more efficient and effective manufacturing process. In embodiments where the absorbent matrix is a single piece single material, a support layer may not be necessary (and assuming the material chosen for the absorbent matrix is suitably stable). The base layer may be absorbent or non-absorbent, and may be rigid or flexible. In some embodiments, the base layer may also be absorbent and be in fluid communication with the absorbent matrix, such that the flow path of liquid sample passes through both the absorbent matrix and the base layer. The base layer can be composed of the absorbent materials as mentioned previously, or it can be composed of a flexible or rigid material such as glass, polymer, or other non-porous or non-absorbent materials as would be understood by those skilled in the art.

The flow path through the absorbent matrix generally includes at least two regions, where the first region is a sample application region, and the second region is a detection region. The sample application region can contain a buffer for solubilizing the sample, or can be simply a location on the absorbent matrix for the application of a liquid sample, but it also can contain other reagents. In some embodiments, the detection region is be downstream of the sample application region, such that the liquid sample, when applied to the application region, travels through at least a portion of the absorbent matrix before reaching the detection region. In other embodiments, the detection region is a third region, and the liquid sample may travel past the detection region to a second region, and subsequently return back to the detection region. In still other embodiments, detection region may be the same as the application region, or at least partially overlap with the application region. In still other embodiments, the detection region may be a circular ring around the application region, such that a liquid sample radiates out from the application region in multiple directions to the detection region ring. It should be appreciated that any number of regions may be designated within the flow path of the liquid sample.

The absorbent matrix contains at least a bioactive sPLA2 substrate and a label, such as a colloidal gold particle. The bioactive sPLA2 substrate and label are each located in one of the aforementioned regions. For example, in one embodiment, the bioactive sPLA2 substrate is located in the first region, or application region, and the label is located in the second region, or detection region. In other embodiments, label is located in the first region, or application region, and the bioactive sPLA2 substrate is located in the second region, or detection region. It should be appreciated that neither the bioactive sPLA2 substrate nor the label are required to be positioned in any particular location of the absorbent matrix. In fact, all that is required is that the bioactive sPLA2 substrate and label are positioned in a location that is within the flow path of the liquid sample. As contemplated herein, the bioactive sPLA2 substrate may be any substrate hydrolysable by sPLA2 enzyme to produce a byproduct that can bind to, or be bound by, a label or a linker that binds to or can be bound by another linker or a label, as would be understood by those skilled in the art. For example, the bioactive sPLA2 substrate may be diheptanoyl thio-PC (1,2-bis(heptanoylthio)glycerophosphocholine) (Caymen Chemical). As contemplated herein, the label may be any label suitable for producing a visible signal, and can bind to, or be bound by, a bioactive sPLA2 substrate byproduct or a linker that binds to or can be bound by another linker or a bioactive sPLA2 substrate byproduct, as would be understood by those skilled in the art. For example, and without limitation, the label may be a gold sol, a fluorescent dye, a water soluble dye, a magnetic labeled particle, or any other convenient label as would be understood by those skilled in the art. When using a gold sol, the gold particles are red in color due to localized surface plasmon resonance, thereby producing a visual, color indicator. In certain embodiments, using a gold sol, aggregation produced a color shift from pink to red to blue, depending on size and concentration of the particles. In one embodiment, a mean particle size of gold particles is about 40 nM. The visually observed color of colloidal gold particles is generally dependent upon the particle size. For example, particles up to about 100 nm in size exhibit an intense red color while particles greater than about 100 nm in size exhibit a somewhat more muted color. Thus, while it is possible according to the invention to use gold particles greater than about 100 nm in size, in preferred embodiments, the test device of the present invention preferentially uses a mean particle size of from about 20 nm to about 60 nm. Preferably, the gold particles used according to the present invention are substantially spherical in shape. However, other shapes could also be used.

In some embodiments, the label may be conjugated with another molecule, such as streptavidin, to better assist in label mobility through the absorbent matrix, label binding, or both. In still another embodiment, a biotin-linker molecule conjugate can be added to the absorbent matrix to amplify the aggregation of streptavidin-conjugated gold particles. In one non-limiting example, the linker molecule is biotin-maleimide (Invitrogen). Further, in such embodiments, a blocker can also be added to the absorbent matrix, such as L-cysteine, to quench biotin-maleimide reactivity. It should be appreciated that any linkers or blockers can be added to the absorbent matrix, as would be understood by those skilled in the art.

While not required, it is preferable for any bioactive sPLA2 substrate, label, linker, blocker or other additive to be dried into or onto the absorbent matrix. Keeping the device dry may provide for better product storage and shelf life. These drying steps can be air dried, vacuum dried, or other drying technique as would be understood by those skilled in the art. Further, these drying steps can be done simultaneously for all or some components, or can be performed separately during construction of the device. In some embodiments, separate absorbent matrix materials having any one or more of these components dried in or on them can be prefabricated, and these prefabricated, laced materials can then be used to construct the absorbent matrix of the device.

Also, the device may optionally include a control region that is separate from the detection region, such that the control region provides a detectable signal. In one embodiment, the control region is a positive control. It should be appreciated that any secondary molecule, wash or other step may be used to visibly detect activity in the control region, as would be understood by those skilled in the art. The positive control region may in another embodiment contain dried purified sPLA2 (either type Ia, from cobra, or Ib from pancreas (both available from Sigma Aldich) which is placed distal to the primary reaction zone intermingled with colloidal gold of the same variety as the primary reaction. The flow of the sample carries the sPLA2 to a second reaction zone containing the substrate in Ca²⁺ buffer as in the first zone. The second zone has a reaction, the color of which is predetermined by adjusting sPLA2 concentration.

### Testing Sample

The sample can be any fluid sample, e.g., a biological sample such as a bodily fluid that is likely to contain sPLA2. Alternatively, the sample may be a solid biological sample that is separately prepared into a fluid sample by mechanical disruption and/or the addition of a fluid medium, such as a buffered solution. In one embodiment, the biological sample is a blood, plasma, serum, saliva, mucus, urine, cervical mucus, cell extracts or amniotic fluid sample. In another embodiment, the biological sample is a urine sample. In another embodiment, the sample is not a biological sample, but a fluid in which, for example, sPLA2 is to be detected. The sample may, but need not be treated prior to being deposited on the test strip. In certain cases where the sample is too viscous to flow evenly on the test strip, the sample may be pre-treated with agents that reduce the viscosity of the fluid, including, but not limited to, one or more mucolytic agents or mucinases.

### Molecular Mechanism of the Device

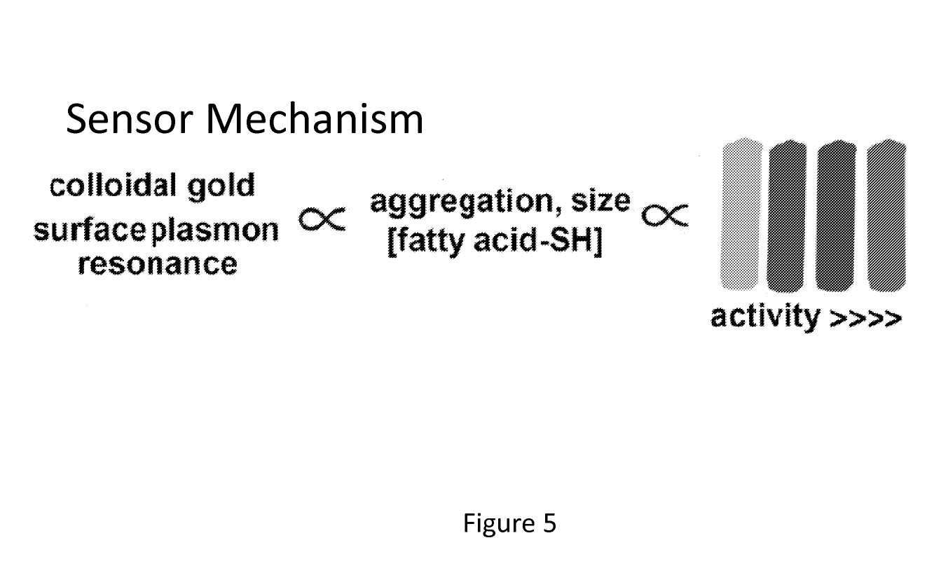

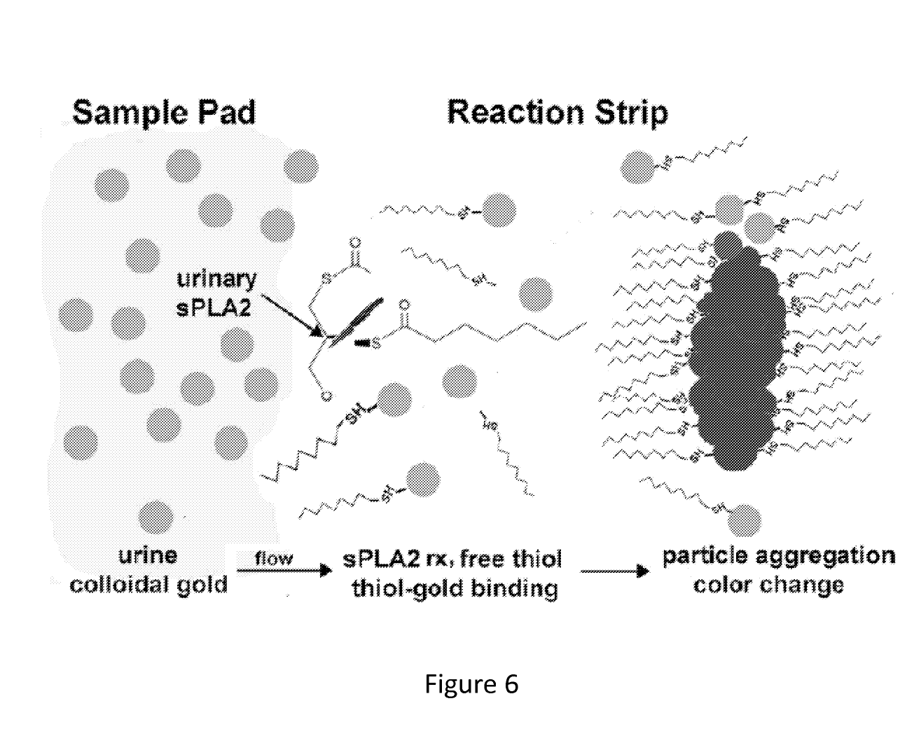

In operation, a sample containing sPLA2 enzyme is added to the sample loading region and flows along a path through the absorbent matrix. As illustrated in FIG. 6, the enzyme cleaves the A2 fatty acid bond, releasing fatty acid thiols that attach to the label. These labeled fatty acid thiols aggregate within the detection region, where they undergo a color change based on the size and concentration of the labeled fatty acid aggregation, and thereby corresponding to a level of enzyme activity. When colloidal gold is used, a lower activity is indicated by a pink color, while a higher activity is indicated by a darker red or blue color. For example, the portion of the visible spectrum of the light reflected from the gold particles depends on surface plasmon resonance as depicted in FIG. 5. In one embodiment, the visually observed color of colloidal gold particles depends on the size of gold particles. In another embodiment, the visually observed color depends on the extent the particles aggregated. In yet another embodiment, the visually observed color depends on concentration of hydrolyzed substrate. In yet another embodiment, the visually observed color depends on the concentration of sPLA2 in the sample. In one aspect the device can be designed to report an atypically high sPLA2 activity by pre-selection of the components, such as colloidal gold particle size, concentration, substrate concentration. In another aspect the device can be designed to grade color change from pink to blue along with a reference chart showing the general level of activity suggested by the color of the line. Without wishing to be bound by any particular theory, it is believed that the amount of fatty acid cleaved from the bioactive sPLA2 substrate (enzyme reaction product) will dictate the extent of aggregation. In certain aspect, the amount of fatty acid cleaved from the sPLA2 substrate shows linearity relation to the extent of gold particle aggregation.

If the label is located in the sample loading region, the label is mobilized when sample is added, and travels with the liquid sample to the detection region, where the bioactive sPLA2 substrate is located. If the bioactive sPLA2 substrate is located in the sample loading region, then the fatty acid thiols are cleaved and mobilized within the liquid sample flow path and into the detection region, where the label is located. In some instances, the device may include three regions, where the first region is the sample loading region and contains one of the bioactive sPLA2 substrate or the label, the second region contains the other of the bioactive sPLA2 substrate or the label, and the third region is the detection region, which may be positioned between the first and second regions, or it may be downstream of the first and second regions.

The technique of the present invention employs a variety of reagents for detecting activity of a protein. One such reagent is a substrate that can be cleaved by a corresponding active protein. The substrate is chemically acted upon or “cleaved” by the protein of interest to release a portion of the substrate where the portion can be detected. In one embodiment, the cleaved substrate is a chromogenic product. The chromogenic product thus released is capable of conversion to a secondary product that is then recognizable. For example, the released chromogenic product may react with a first reagent to form a second reagent that has a discernable color.

In one embodiment, the invention is generally directed to a lateral flow assay device for detecting the presence or quantity of an enzyme. The assay device utilizes a molecular substrate such as, for example, a peptide, protein, or glycoprotein substrate, to facilitate the detection of the enzyme. The molecular substrate provides a target for an enzyme, such as a proteolytic enzyme. Specifically, upon contacting the molecular substrate, a proteolytic enzyme cleaves the molecular substrate and releases an enzyme reaction product. The assay device also utilizes a detectable substance that may generate a detection signal upon reaction of an enzyme with the molecular substrate. The signal generated by the detectable substance may then be used to indicate the presence or quantity of an enzyme within a test sample.

Various types of enzymes may be detected in accordance with the present disclosure. For instance, transferases, hydrolases, lyases, and so forth, may be detected. In some embodiments, the enzyme of interest is a “hydrolase” or “hydrolytic enzyme”, which refers to enzymes that catalyze hydrolytic reactions. Examples of such hydrolytic enzymes include, but are not limited to, proteases, peptidases, lipases, nucleases, homo- or hetero-oligosaccharidases, homo- or hetero-polysaccharidases, phosphatases, sulfatases, neuraminidases and esterases. In one embodiment, for example, peptidases may be detected. “Peptidases” are hydrolytic enzymes that cleave peptide bonds found in shorter peptides. Examples of peptidases include, but are not limited to, metallopeptidases; dipeptidylpeptidase I, II, or IV; and so forth. In another embodiment, proteases may be detected. “Proteases” are hydrolytic enzymes that cleave peptide bonds found in longer peptides and proteins. Examples of proteases that may be detected include, but are not limited to, serine proteases (e.g., chymotrypsin, trypsin, elastase, PSA, etc.), aspartic proteases (e.g., pepsin), thiol proteases (e.g., prohormone thiol proteases), metalloproteases, acid proteases, and alkaline proteases.

Without wishing to be bound by any particular theory, any enzyme that is active in inflammation is applicable to the present invention. This is because activity of these enzymes using the device of the present invention is indicative of an inflammatory response in the mammal from which the biological sample being tested is derived from.

As discussed elsewhere herein, molecular substrates may be used to detect the presence or quantity of an enzyme. The molecular substrate may occur naturally or be synthetic. Some suitable molecular substrates for hydrolytic enzymes include, for instance, esters, amides, peptides, ethers, or other chemical compounds having an enzymatically-hydrolyzable bond. The enzyme-catalyzed hydrolysis reaction may, for example, result in a hydroxyl or amine compound as one product, and a free phosphate, acetate, etc., as a second product. Specific types of molecular substrates may include, for instance, proteins or glycoproteins, peptides, nucleic acids (e.g., DNA and RNA), carbohydrates, lipids, esters, derivatives thereof, and so forth. For instance, some suitable molecular substrates for peptidases and/or proteases may include peptides, proteins, and/or glycoproteins, such as casein (e.g., β-casein, azocasein, etc.), albumin (e.g., bovine serum albumin (BSA)), hemoglobin, myoglobin, keratin, gelatin, insulin, proteoglycan, fibronectin, laminin, collagen, elastin, and so forth. Still other suitable molecular substrates are described in U.S. Pat. No. 4,748,116 to Simonsson, et al.; U.S. Pat. No. 5,786,137 to Diamond, et al.; U.S. Pat. No. 6,197,537 to Rao, et al.; and U.S. Pat. No. 6,235,464 to Henderson, et al.; U.S. Pat. No. 6,485,926 to Nemori, et al., which are incorporated herein in their entirety by reference thereto for all purposes.

Following contact of a molecular substrate with an enzyme, an enzyme reaction product may form. The molecule substrate or the enzyme reaction product may than interact with a detectable substance so as to directly or indirectly generate a detectable signal. Suitable detectable substances may include, for instance, chromogens; luminescent compounds (e.g., fluorescent, phosphorescent, etc.); radioactive compounds; visual compounds (e.g., latex or metallic particles, such as gold); liposomes or other vesicles containing signal-producing substances; enzymes and/or substrates, and so forth. For instance, some enzymes suitable for use as detectable substances are described in U.S. Pat. No. 4,275,149 to Litman, et al., which is incorporated herein in its entirety by reference thereto for all purposes. One example of an enzyme/substrate system is the enzyme alkaline phosphatase and the substrate nitro blue tetrazolium-5-bromo-4-chloro-3-indolyl phosphate, or derivative or analog thereof, or the substrate 4-methylumbelliferyl-phosphate. Other suitable detectable substances may be those described in U.S. Pat. No. 5,670,381 to Jou, et al. and U.S. Pat. No. 5,252,459 to Tarcha, et al., which are incorporated herein in their entirety by reference thereto for all purposes.

Following contact, any enzyme present within the test sample will typically interact with at least a portion of the substrate molecules. As a result, various species may be formed, including enzyme reaction products, partially cleaved complexes (e.g., enzyme-substrate complexes), unreacted substrate molecules, and secondary reactants and products of the enzyme-catalyzed reaction. For instance, in the case of a hydrolytic enzyme, at least two products (which may be the same or different) formed during the enzyme-catalyzed cleavage of the substrate molecule will be included in the mixture. When considering an enzyme-catalyzed reaction in which new bonds are formed on the substrate, materials included in the mixture may include other reactants involved in the reaction (e.g., ATP, methyl-donating reactants, monomers such as amino acids, and nucleotides that may be added to the substrate by a polymerase or a ligase, etc.) as well as secondary products formed in the enzyme-catalyzed reaction (e.g., ADP).

Methods of Monitoring sPLA2 Activity and Disease

The present invention further relates to a method of determining the presence or absence of sPLA2 in a sample. Typically, the method comprises adding a liquid sample to any of the devices as described herein, allowing the liquid sample to flow along the flow path to form a detectable reaction product when sPLA2 is in the sample, and observing the detection region of the device to determine the presence or absence of a detectable reaction product. As contemplated herein, the aggregated, labeled bioactive sPLA2 substrate byproduct is the detectable reaction product.

The present invention also relates to a method of determining a variable amount of sPLA2 in a sample, or a variable level of sPLA2 activity in a sample. The method comprises adding a liquid sample to any of the devices as described herein, allowing the liquid sample to flow along the flow path to form a visibly detectable reaction product when sPLA2 is in the sample, observing the detection region of the device to determine at least one of the color or intensity of the visibly detectable reaction product, and comparing at least one of the color or intensity of the visibly detectable reaction product to a predetermined set of colors or intensities, wherein each of the predetermined colors or intensities is indicative of an amount of sPLA2 in the sample, or of a level of sPLA2 activity in the sample. As contemplated herein, the aggregated, labeled bioactive sPLA2 substrate byproduct is the visibly detectable reaction product.

The present invention also relates to a method of determining a pathology, a disease or response to treatment of a disease. The method comprises adding a liquid sample collected from a patient to any of the devices as described herein, allowing the liquid sample to flow along the flow path to form a visibly detectable reaction product when sPLA2 is in the sample, observing the detection region of the device to determine at least one of the color or intensity of the visibly detectable reaction product, and comparing at least one of the color or intensity of the visibly detectable reaction product to a predetermined set of colors or intensities, wherein selected colors or intensities of the predetermined colors or intensities is indicative of a pathology, a disease, or of a category of pathology or disease in the patient. As contemplated herein, the aggregated, labeled bioactive sPLA2 substrate byproduct is the visibly detectable reaction product. As previously described types of diseases suitable for such monitoring include (without limitation) multiple sclerosis, rheumatoid arthritis, atherosclerosis and Alzheimer's disease. It should be appreciated that the aforementioned methods are suitable for monitoring any pathology or disease, or treatment to such pathology or disease, where increased sPLA2 activity in the sample is indicative of the pathology or disease in the patient.

The invention should not be limited to only detecting sPLA2 activity. Rather, the invention includes detecting any PLA2 including cytosolic phospholipases A2 (cPLA2) and lipoprotein-associated PLA2s (1p-PLA2). Due to the importance of PLA2 in inflammatory responses, detection of PLA2 activity is an indication of the level of inflammatory response.

The invention also provides compositions and method of detecting inflammation wherein inflammation is characteristic of increased activity of a protein to cleave a corresponding substrate. The invention includes a device for detecting activity of a protein to cleave a substrate wherein cleavage of the substrate allows the substrate to be detected. An increased detection of the substrate is an indication of the presence of the active protein and thereby indication of an inflammatory response.

### Diagnostic Kit

The present invention also includes a diagnostic kit for monitoring levels of sPLA2 activity. The test kit comprises any one of the devices as described herein, and an instruction manual providing instruction of how to use and determine any results indicated by the device. The kit may also include a reference chart of various colors or intensities, wherein each of the colors or intensities is indicative of an amount of sPLA2 in the sample, or of a level of sPLA2 activity in the sample.

### EXAMPLES

The invention is further described in detail by reference to the following examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the devices of the present invention and practice the described methods. The following working examples therefore, identify exemplary embodiments of the present invention, and are not to be construed as limiting in any way the remainder of the disclosure.

### Example 1

### Devices Having Three Regions

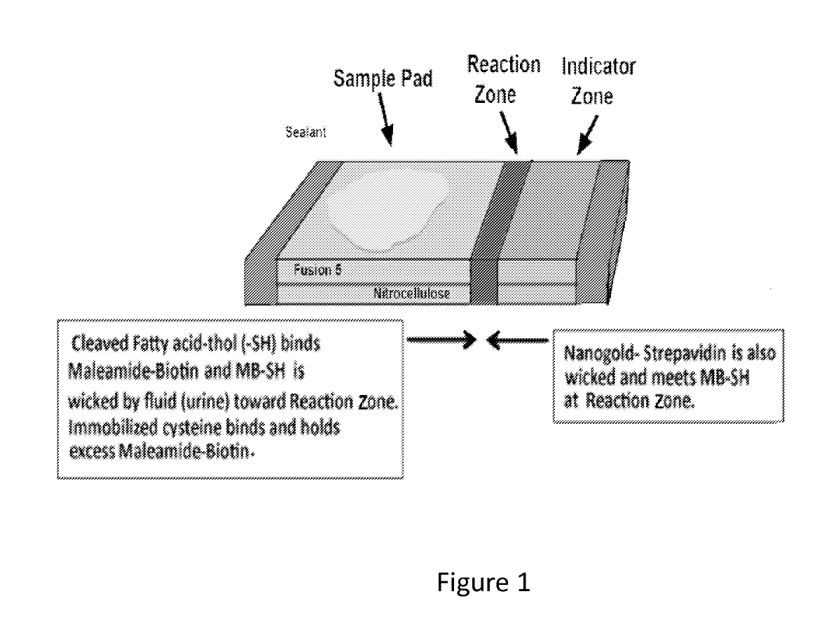

A device for monitoring levels of sPLA2 activity having three regions was created and is depicted in FIG. 1. The device includes a bottom or base layer of nitrocellulose paper NC90 (Millipore) and a top layer of Fusion 5 paper (Whatman), with the ends sealed by rubber cement. The device includes three designated regions. The first region, which is also the sample application region, includes dried bioactive sPLA2 substrate known as Diheptanoyl Thio-PC (Caymen Chemical), a dried linker molecule biotin-maleimide (Invitrogen), and a dried blocking compound L-cysteine-agarose (Sigma). The second region includes a dried colloidal gold particle covalently linked to streptavidin, known as Nanogold (Bioassayworks, DSTAL-B001). The third region is a detection zone located between the first and second regions. Diheptanoyl Thio-PC was selected as the substrate because it is a substrate for all phospholipase A2s (PLA2s) with the exception of cPLA2 and PAF-acetyl hydrolase (PAF-AH). Interaction of this compound with a sPLA2 results in cleavage of the sn2 fatty acid with a free thiol attached. Biotin-maleimide is a maleimide group covalently linked to a biotin. This linker was selected because the maleimide binds the free thiol attached to the fatty acid and can be wicked toward the downstream regions after addition of a urine sample. L-cysteine-Agarose (Sigma) was selected as a blocker, because immobilized cysteine (—SH) binds and holds the unreacted biotin-maleimide. Similar results were obtained by adding cysteine solution directly, suggesting the biotin-maleimide bound to cys is less mobile than that bound to the thiol-fatty acid, possibly because of aggregation of the former complex. Stepavidin conjugated to gold particles was selected as the label because it produces a color shift of pink to red to blue upon aggregation.

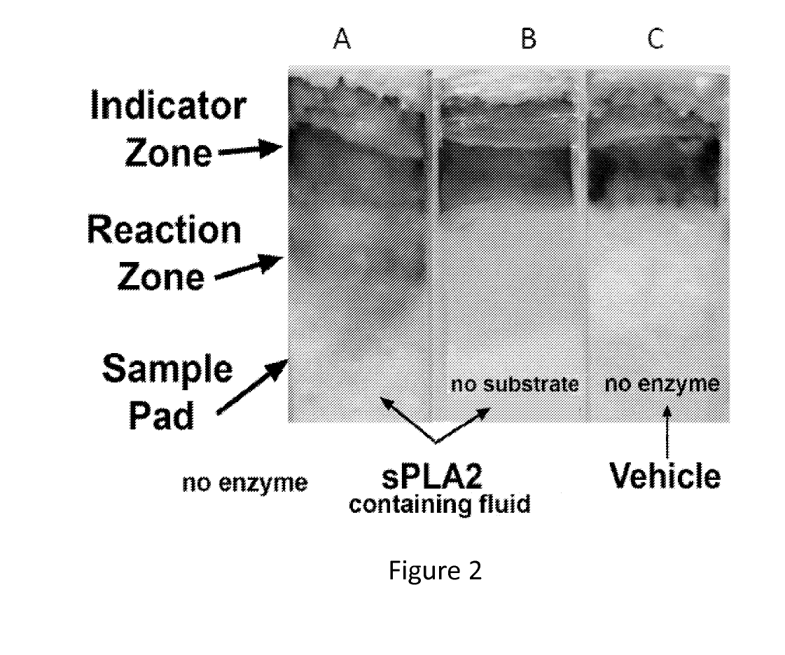

As demonstrated in FIG. 2, three such devices (2A-C) were constructed according to the embodiment of FIG. 1. For each of devices 2A-C, the first region was infused with 10 μl of a mixture of 25% 48 mM bis-thioPC substrate in 10 mM Tris-150 mM NaCl buffer, pH=7.4 that contained 4 mM CaCl₂ and 10 μg maleimide-biotin. Care was taken to restrict fluid to the first region area only. The pads were dried 15 min under vacuum. Cysteine solution (7 μl, 1.5 mM) was then added to the first region and air dried. Nanogold (40 nM, optical density 15) was used without dilution and 2.5 μl was delivered in 0.5 μl spots in a strip across the second region of each device 2A-C. The paper for each was air-dried and the procedure was repeated twice. A first control strip (2B) was prepared in which the substrate's vehicle was used instead of substrate (no substrate), and a second control strip (2C) was prepared in which the enzyme's vehicle was added during the test instead of enzyme (no enzyme). The samples for devices 2A and 2B were 5×10⁻⁸M sPLA2 added as two large drops over the first region. The sample for 2C only contained the enzyme vehicle. As seen in FIG. 2, dark blue reaction appeared in the third region only when enzyme and substrate are both present as depicted in device 2A.

### Example 2

### Devices Having Two Regions

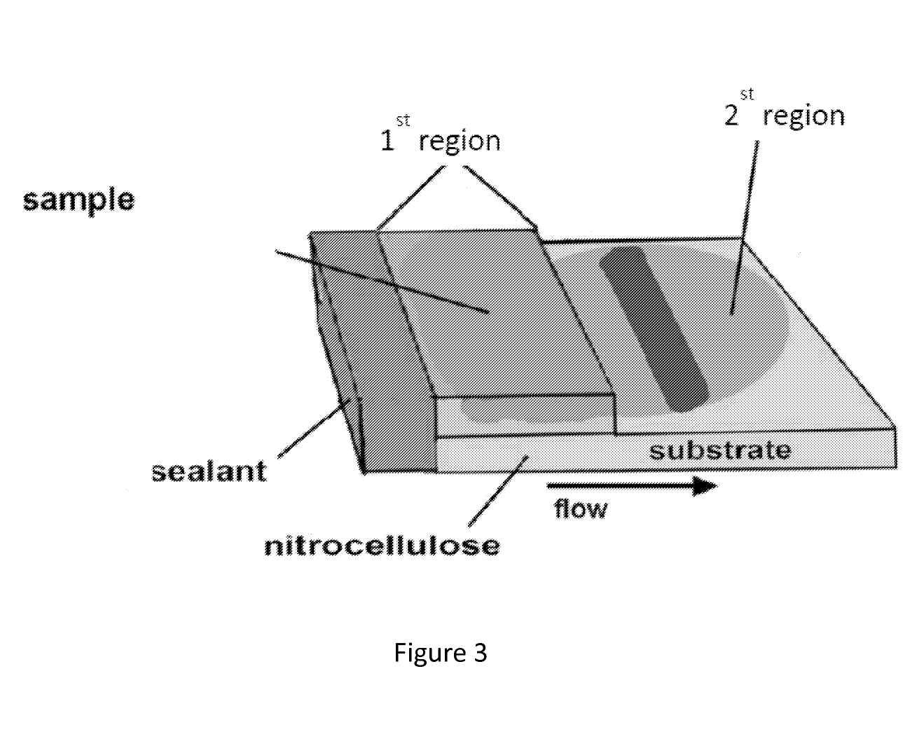

A device for monitoring levels of sPLA2 activity having two regions was created and is depicted in FIG. 3. The device includes a bottom or substrate layer of nitrocellulose NC90 and a top layer of Whatman Fusion 5 paper covering a portion of the base layer, with the end sealed by rubber cement. The device includes two designated regions. The first region (the top layer of Whatman Fusion 5 paper) was spotted with colloidal gold (40 nM, optical density (OD) 15 used without dilution, or OD 50 used at 25% in water) by delivering about 10-15 μl in 1 μl spots over the entire region, and then air-dried. The second region (exposed substrate layer) was spotted with 10 μl of a mixture of 25% 48 mM bis-thio PC substrate in 10 mM Tris-150 mM NaCl buffer, pH=7.4 that contained 4 mM CaCl₂, and vacuum dried.

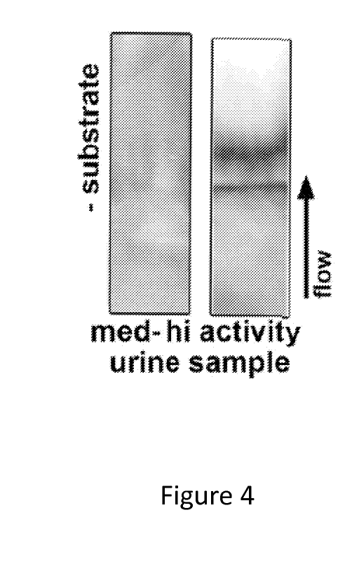

As demonstrated in FIG. 4, two devices (4A and 4B) were constructed according to the embodiment of FIG. 3. The size of each device was about 1.5×3 cm. One drop of human urine was added to the first region of each device 4A and 4B from a Pasteur pipette. As shown in FIG. 4, dark red lines appeared in the second region and a thin line appeared at the border with the first region of device 4B. The control strip (4A) without substrate is also shown and lacks the aggregated reaction product seen in the second region of device 4B. Controls without enzyme were also non-reactive.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A device for detecting the presence or absence of sPLA2 in a fluid sample, comprising:
an absorbent matrix that defines a flow path for a fluid sample; a first region of the absorbent matrix for applying a fluid sample, wherein one of the components selected from a bioactive sPLA2 substrate and a label is dried onto or within the first region of the absorbent matrix; and
a second region of the absorbent matrix downstream of, and in fluid communication with, the first region for detecting an aggregated reaction product, wherein the other component not present in the first region is dried onto or within the second region of the absorbent matrix;
wherein in the absence of sPLA2 in the fluid sample, applying the fluid sample does not result in a recognizable aggregated reaction product in the second region; and
wherein in the presence of sPLA2 in the fluid sample, applying the fluid sample results in a detectable aggregated reaction product in the second region.

2. The device of claim 1, wherein the label comprises a gold sol.

3. The device of claim 2, wherein the gold sol comprises streptavidin coated gold particles.

4. The device of claim 1, wherein the bioactive sPLA2 substrate is Diheptanoyl Thio-PC.

5. The device of claim 3, further comprising a linker molecule dried onto or within the absorbent matrix in the same region as the bioactive sPLA2 substrate.

6. The device of claim 5, wherein the linker molecule is biotin-maleimide.

7. The device of claim 6, wherein a blocker molecule is dried onto or within the absorbent matrix in the same region as the linker molecule.

8. The device of claim 1, wherein the fluid is a biological sample.

9. The device of claim 8, wherein the biological sample is urine.

10. A device for determining the level of sPLA2 activity in a fluid sample, comprising:
an absorbent matrix that defines a flow path for a fluid sample; a first region of the absorbent matrix for applying a fluid sample, wherein one of the components selected from a bioactive sPLA2 substrate and a label is dried onto or within the first region of the absorbent matrix;
a second region of the absorbent matrix downstream of, and in fluid communication with, the first region for detecting an aggregated reaction product, wherein the other component not present in the first region is dried onto or within the second region of the absorbent matrix;
wherein after applying a fluid sample containing sPLA2 to the first region, one of the color or intensity of the aggregated reaction product can be compared to a predetermined set of colors or intensities, wherein each of the predetermined colors or intensities is indicative of a level of sPLA2 activity in the fluid sample.

11. The device of claim 10, wherein the label comprises a gold sol.

12. The device of claim 11, wherein the gold sol comprises streptavidin coated gold particles.

13. The device of claim 10, wherein the bioactive sPLA2 substrate is Diheptanoyl Thio-PC.

14. The device of claim 13, further comprising a linker molecule dried onto or within the absorbent matrix in the same region as the bioactive sPLA2 substrate.

15. The device of claim 14, wherein the linker molecule is biotin-maleimide.

16. The device of claim 15, wherein a blocker molecule is dried onto or within the absorbent matrix in the same region as the linker molecule.

17. The device of claim 10, wherein the fluid sample is a biological sample.

18. The device of claim 17, wherein the biological sample is urine.

19. A device for determining the level of sPLA2 activity in a fluid sample, comprising:
an absorbent matrix that defines a flow path for a fluid sample; a first region of the absorbent matrix for applying a fluid sample, wherein one of the components selected from a bioactive sPLA2 substrate and a label is dried onto or within the first region of the absorbent matrix;
a second region of the absorbent matrix downstream of, and in fluid communication with, the first region, wherein the other component not present in the first region is dried onto or within the second region of the absorbent matrix; and
a third region of the absorbent matrix downstream of the first region, and in fluid communication with the first and second regions, for detecting an aggregated reaction product;
wherein after applying a fluid sample containing sPLA2 to the first region, the liquid sample mobilizes the components of the first and second regions and forms an detectable aggregation product in the third region,
and wherein one of the color or intensity of the aggregated reaction product can be compared to a predetermined set of colors or intensities, wherein each of the predetermined colors or intensities is indicative of a level of sPLA2 activity in the fluid sample.

20. A method of determining the presence or absence of sPLA2 in a fluid sample, comprising:
adding a fluid sample to the device of claim 1;
allowing the fluid sample to flow along the flow path to form a detectable aggregated reaction product in the second region when sPLA2 is in the fluid sample; and
observing the second region of the device to determine the presence or absence of a detectable aggregated reaction product.

21. (canceled)

22. (canceled)
